(19) United States
(12) Patent Application Publication
(10) Pub. No.: US 2024/0164949 A1
(43) Pub. Date: May 23, 2024

(54) Modular Medical Dressing

(71) Applicant: Becton, Dickinson and Company, Franklin Lakes, NJ (US)

(72) Inventors: Manish Kumar, Bengaluru (IN); Shishir Prasad, Ramsey, NJ (US); Praveen Nalawade, Belagavi (IN); Aashish Ahuja, Durham, NC (US)

(21) Appl. No.: 17/993,122

(22) Filed: Nov. 23, 2022

(51) Int. Cl. A61F 13/02

(52) U.S. Cl. CPC A61F 13/023; A61F 13/0246

(57) ABSTRACT

A modular medical dressing includes a layered dressing arrangement having a dressing layer with a first surface and a second surface, a release layer releasably adhered to the first surface, and a backing layer releasably adhered to the second surface. The modular medical dressing further includes a pre-defined tearable portion formed on the layered dressing arrangement. The pre-defined tearable portion is tearable from the layered dressing arrangement to reduce a surface area of the layered dressing arrangement. The pre-defined tearable portion may have a plurality of first tear lines connected to define a first tearable portion. At least a portion of the release layer is configured for use as securement tape for securing a catheter to an anatomical structure.

### BACKGROUND

### Technical Field

The present disclosure relates to modular medical dressing. More specifically, the present disclosure relates to modular medical dressing having a layered dressing arrangement with a pre-defined tearable portion tearable from the layered dressing arrangement to reduce a surface area of the layered dressing arrangement.

### Technical Considerations

There is an increasing shift in medicine towards remote patient monitoring and treatment. Intravenous (IV) therapy remains one of the most widely used methods of providing such remote monitoring and care to the patient. IV therapy may refer to a medical technique that is used to deliver fluids, medications, and/or nutrition directly into a body of a patient via an IV catheter inserted into a vein at an IV insertion site. IV therapy may be used for rehydration, to provide nutrition to a person who cannot consume food or water by mouth, and/or to administer medications or other medical therapy, such as blood products or electrolytes.

After an IV catheter is inserted at an IV insertion site, the IV catheter must be secured from being accidentally pulled. Typically, the IV catheter is secured at the IV insertion site via an adhesive, such as an adhesive tape, that attaches directly to the patient's skin. A medical dressing is then applied to an area surrounding the IV insertion site to prevent catheter contamination and keep the IV insertion site clean.

Healthcare providers must stock medical dressings in various sizes to accommodate patients of various sizes, ranging from infants to adults. Additionally, healthcare providers must separately stock securement tape in order to secure the IV catheter in place. There is an increasing need for improved medical dressings that overcome the deficiencies of the existing medical dressings.

### SUMMARY

In some non-limiting embodiments or aspects, provided is a modular medical dressing that may include a layered dressing arrangement having a dressing layer with a first surface and a second surface; a release layer releasably adhered to the first surface; and a backing layer releasably adhered to the second surface. The modular medical dressing further may include a pre-defined tearable portion formed on the layered dressing arrangement. The pre-defined tearable portion may be tearable from the layered dressing arrangement to reduce a surface area of the layered dressing arrangement.

In some non-limiting embodiments or aspects, the pre-defined tearable portion may have a plurality of first tear lines connected to define a first tearable portion. The pre-defined tearable portion further may have a plurality of second tear lines spaced apart from the plurality of first tear lines and connected to define a second tearable portion. The pre-defined tearable portion may be a perforation extending through at least a portion of each of the dressing layer, the release layer, and the backing layer.

In some non-limiting embodiments or aspects, the dressing layer may be transparent. The dressing layer may have a catheter access slot. The second surface of the dressing layer may have an adhesive for adhering the dressing layer to an anatomical structure. The dressing layer may be made from a polyurethane material.

In some non-limiting embodiments or aspects, the backing layer may be made from a paper material. The release layer may be made from a paper material and may include an adhesive on a surface facing the first surface of the dressing layer. At least a portion of the release layer may be configured for use as securement tape for securing a catheter to an anatomical structure.

In some non-limiting embodiments or aspects, a modular medical dressing may have a layered dressing arrangement with a dressing layer having a first surface and a second surface and a release layer releasably adhered to the first surface; and a pre-defined tearable portion formed on the layered dressing arrangement. The pre-defined tearable portion is tearable from the layered dressing arrangement to reduce a size of the layered dressing arrangement.

In some non-limiting embodiments or aspects, the modular medical dressing may include a backing layer made of paper and releasably adhered to the second surface of the dressing layer.

In some non-limiting embodiments or aspects, the pre-defined tearable portion may have a plurality of first tear lines connected to define a first tearable portion. The pre-defined tearable portion further may include a plurality of second tear lines spaced apart from the plurality of first tear lines and connected to define a second tearable portion. The pre-defined tearable portion may be a perforation extending through at least a portion of each of the dressing layer, the release layer, and the backing layer.

In some non-limiting embodiments or aspects, the dressing layer may be transparent. The second surface of the dressing layer may have an adhesive for adhering the dressing layer to an anatomical structure. The release layer may be made from a paper material and may include an adhesive on a surface facing the first surface of the dressing layer. A portion of the release layer may be configured for use as securement tape for securing a catheter to an anatomical structure.

### BRIEF DESCRIPTION OF THE DRAWINGS

Additional advantages and details are explained in greater detail below with reference to the exemplary embodiments that are illustrated in the accompanying schematic figures, in which:

FIG. 1 is a front view of a modular medical dressing in accordance with one embodiment or aspect of the present disclosure;

FIG. 2 is an exploded perspective view of the modular medical dressing shown in FIG. 1;

FIG. 3 is an exploded side view of the modular medical dressing shown in FIG. 1;

FIG. 4 is a perspective view showing a reduction in size of the modular medical dressing shown in FIG. 1 in accordance with one example;

FIG. 5 is a perspective view showing a reduction in size of the modular medical dressing shown in FIG. 1 in accordance with another example;

FIG. 6 is a front view of a release liner of the modular medical dressing shown in FIG. 1 showing a release of a securement tape;

FIG. 7 is a front view of a securement tape in accordance with one example;

FIG. 8 is a front view of a securement tape in accordance with one example; and

FIG. 9 is a perspective view of securement tape in use with an IV catheter in accordance with various examples of the present disclosure.

FIG. 10 is a perspective view of securement tape in use with an IV catheter in accordance with various examples of the present disclosure.

FIG. 11 is a perspective view of securement tape in use with an IV catheter in accordance with various examples of the present disclosure.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided is a modular medical dressing having a layered dressing arrangement with a pre-defined tearable portion tearable from the layered dressing arrangement to reduce a surface area of the layered dressing arrangement. Different sizes of dressings can be obtained from the same layered dressing arrangement by tearing a desired section of the pre-defined tearable portion.

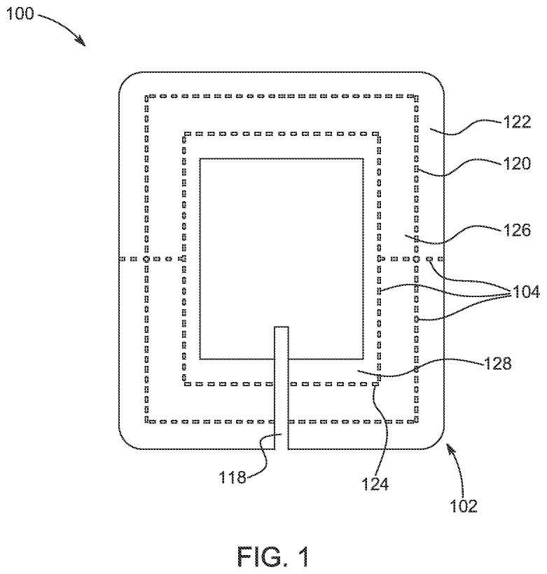

Referring now to FIGS. 1-3, a modular medical dressing 100 is shown in accordance with various embodiments or aspects. The modular medical dressing 100 is configured for placement on an anatomical structure, such as over a bodily appendage of a patient. In various embodiments or aspects, the modular medical dressing 100 is configured to be applied on the bodily appendage near an IV catheter insertion site. For example, the modular medical dressing 100 can be applied on at least a portion of the patient's hand, arm, leg, neck, or other anatomical structure. In some embodiments or aspects, the modular medical dressing 100 is configured for use with an IV catheter (not shown). In further embodiments or aspects, the modular medical dressing 100 may be configured for use with other long term access devices, such as urinary catheters.

With continued reference to FIGS. 1-3, the modular medical dressing has a layered dressing arrangement 102 and a pre-defined tearable portion 104 formed on the layered dressing arrangement 102. The pre-defined tearable portion 104 is tearable from the layered dressing arrangement 102 to reduce a surface area of the layered dressing arrangement 102. In this manner, different sizes of dressings can be obtained from the same layered dressing arrangement 102 by tearing a desired section of the pre-defined tearable portion 104, as described herein.

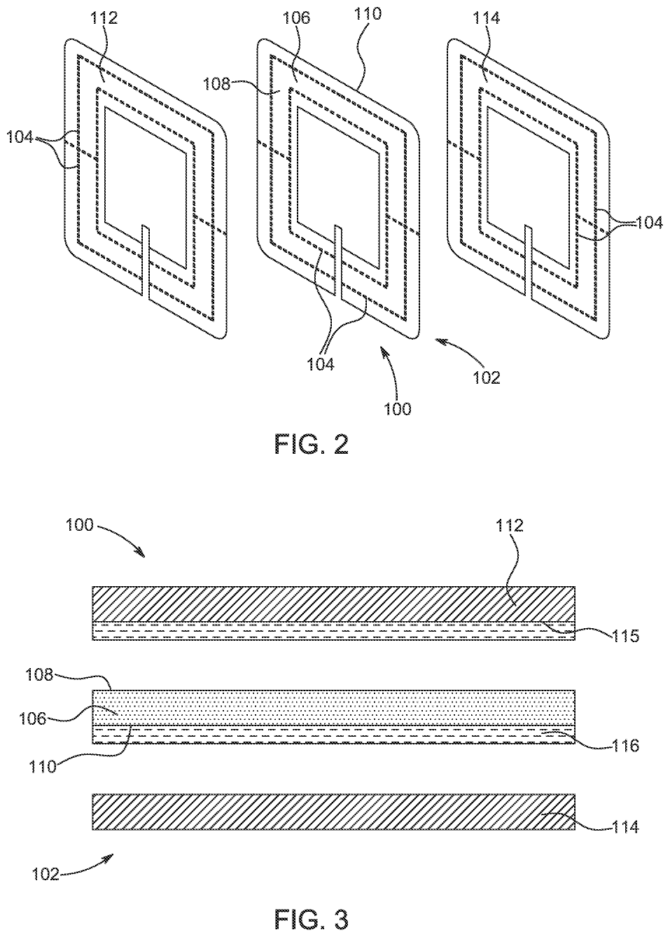

With reference to FIGS. 2-3, the layered dressing arrangement 102 of the modular medical dressing 100 has a stacked arrangement of a plurality of releasably adhered layers. In particular, the layered dressing arrangement 102 has a dressing layer 106 having a first surface 108 and a second surface 110. The dressing layer 106 is disposed between a release layer 112 and a backing layer 114. Specifically, the release layer 112 is releasably adhered to the first surface 108 of the dressing layer 106, while the backing layer 114 releasably adhered to the second surface 110 of the dressing layer 106.

As shown in FIG. 3, the release layer 112 may have a first adhesive 115 for adhering to the first surface 108 of the dressing layer 106. The second surface 110 of the dressing layer 106 may have a second adhesive 116 for adhering to the backing layer 114. The second adhesive 116 may also be configured to adhere to the patient's skin when the backing layer 114 is removed from the dressing layer 106.

With reference to FIG. 2, the layered dressing arrangement 102 may have a substantially rectangular shape. In some embodiments or aspects, a slot 118 may extend from an outer perimeter of the layered dressing arrangement 102. The slot 118 may be configured to receive an IV catheter line (not shown).

With continued reference to FIG. 2, the dressing layer 106 may be a transparent layer configured to permit the IV catheter and/or the IV insertion site to be visible therethrough. In some embodiments or aspects, the dressing layer 106 may be made from a polyurethane material. In further embodiments or aspects, the dressing layer 106 may be made from non-woven material, woven fabric material, or a foam material, such as a silicone foam material.

With continued reference to FIG. 2, the backing layer 114 may be made from a paper material. In some embodiments or aspects, the backing layer 114 may be made from non-woven material, woven fabric material, or a foam material. The backing layer 114 may be sized and shaped to correspond to the shape of the dressing layer 106.

With continued reference to FIG. 2, the release layer 112 may be made from a paper material. In further embodiments or aspects, the release layer 112 may be made from a plastic film material, such as polyester, polyvinyl chloride, polyethylene, or polypropylene film material. As discussed herein, the release layer 112 has the first adhesive 115 for adhering to the first surface 108 of the dressing layer 106. At least a portion of the release layer 112 may be configured for use as a securement tape for securing a catheter to an anatomical structure, as discussed herein.

With continued reference to FIG. 2, the pre-defined tearable portion 104 is formed on each of the layers of the layered dressing arrangement 102. In some embodiments or aspects, the pre-defined tearable portion 104 may be a plurality of perforations extending through at least a portion of each of the dressing layer 106, the release layer 112, and the backing layer 114. In some embodiments or aspects, the perforations may be formed during manufacture of the layered dressing arrangement 102, such as using a roller having a plurality of projections.

The pre-defined tearable portion 104 may have a plurality of first tear lines 120 connected to define a first tearable portion 122. The pre-defined tearable portion 104 further may have a plurality of second tear lines 124 spaced apart from the plurality of first tear lines 120 and connected to define a second tearable portion 126. The first and second tearable portions 122, 126 are removably connected to a main portion 128 of the layered dressing arrangement 102. In this manner, the first and second tearable portions 122, 126 permit the layered dressing arrangement 102 to be reduced in size to fit smaller patients. Having the ability to configure the layered dressing arrangement 102 in 3 different sizes depending on the presence or absence of the first and second tearable portions 122, 126 from the main portion 128 simplifies inventory management for healthcare professionals, who only need to stock a single modular medical dressing 100.

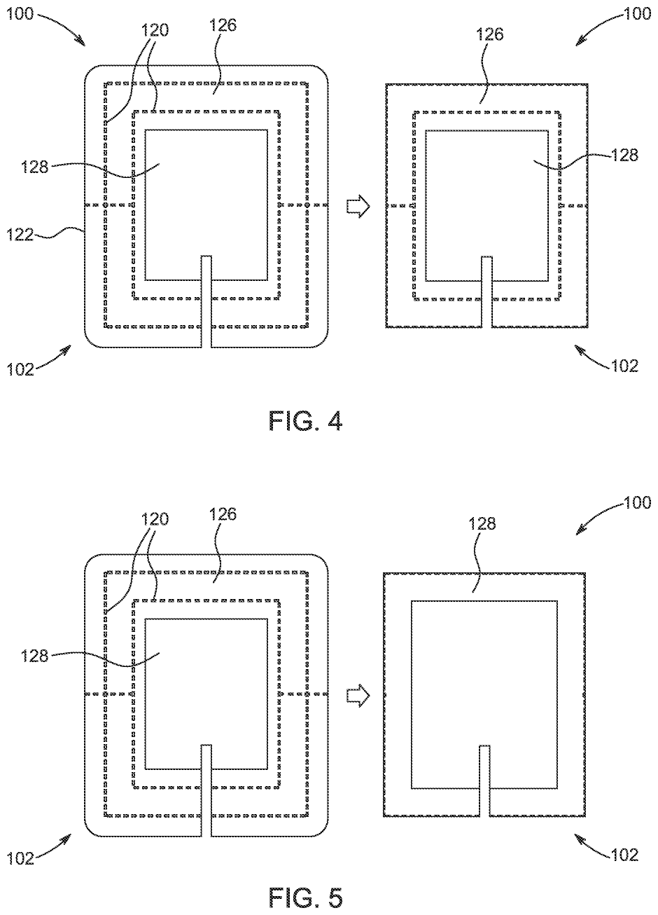

With reference to FIG. 4, in order to reduce the size of the modular medical dressing 100 to an intermediate size, the first tearable portion 122 is removed from the layered dressing arrangement 102 by tearing the first tearable portion 122 along the plurality of first tear lines 120. The backing layer 114 can then be removed from the dressing layer 106 to expose the second adhesive 116 of the dressing layer 106 (shown in FIG. 3) to allow the dressing layer 106 to be applied to the patient's skin. With the dressing layer 106 applied to the patient's skin, the release layer 116 can be removed from the dressing layer 106.

With reference to FIG. 5, in order to reduce the size of the modular medical dressing 100 to the smallest size, the first and second tearable portions 122, 126 are removed from the main portion 128 of the layered dressing arrangement 102 by tearing the first tearable portion 122 along the plurality of first tear lines 120 and the second tearable portion 126 along the plurality of second tear lines 124. In some embodiments or aspects, removal of the second tearable portion 126 from the main portion 128 also removes the first tearable portion 122. The backing layer 114 can then be removed from the dressing layer 106 to expose the second adhesive 116 of the dressing layer 106 (shown in FIG. 3) to allow the dressing layer 106 to be applied to the patient's skin. With the dressing layer 106 applied to the patient's skin, the release layer 116 can be removed from the dressing layer 106.

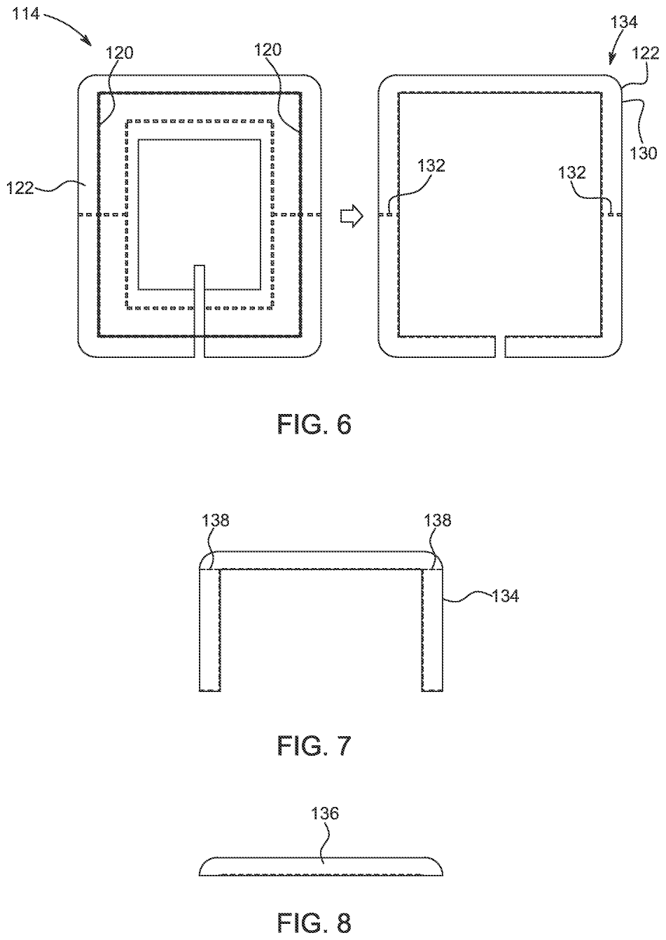

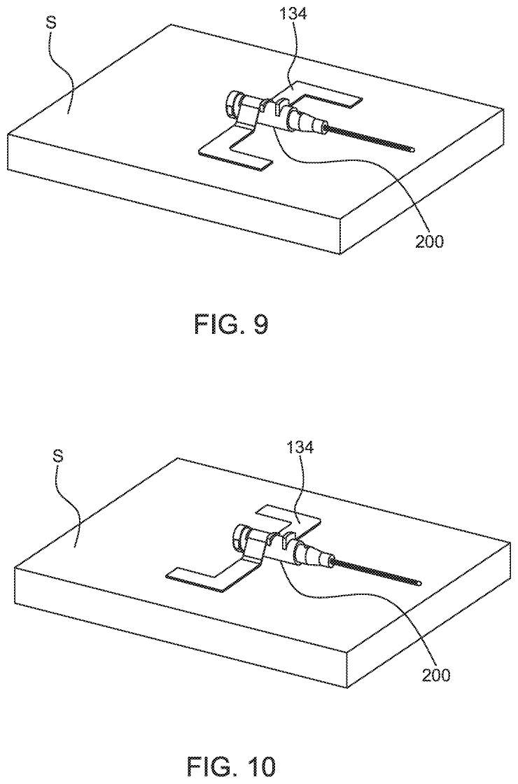

With reference to FIGS. 6-8, the release layer 114 is shown separate from the remaining layers of the layered dressing arrangement 102. Because the release layer 114 has the first adhesive 115, portions of the release layer 114 can be used as securement tape, as discussed herein. As shown in FIG. 6, a first portion 130 of the release layer 114 can be removed by tearing the first tearable portion 122 along the plurality of first tear lines 120. With removal of the first portion 130 of the release layer 114 from the remainder of the release layer 114, the first portion 130 can be split into two U-shaped sections by tearing a pair of first section lines 132. The resulting U-shaped sections define a first securement tape 134 that can be used to secure an IV catheter 200 and/or the dressing layer 106 to the patient's skin (see FIGS. 9-10).

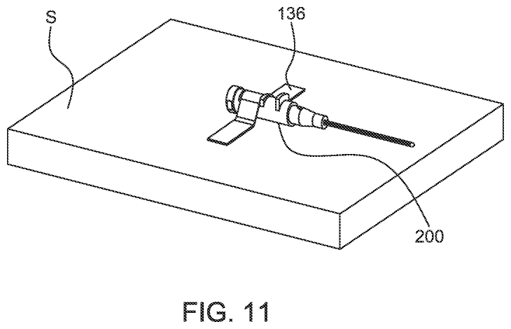

With reference to FIGS. 7-8, the first securement tape 134 can be further split into a plurality of second securement tapes 136 by tearing a pair of second section lines 138. The resulting linear sections define the second securement tape 136 that can be used to secure an IV catheter 200 and/or the dressing layer 106 to the patient's skin (see FIG. 11). In this manner, the healthcare practitioner can choose the size of securement tape based on the patient size.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A modular medical dressing comprising:
a layered dressing arrangement comprising:
a dressing layer having a first surface and a second surface;
a release layer releasably adhered to the first surface; and
a backing layer releasably adhered to the second surface; and
a pre-defined tearable portion formed on the layered dressing arrangement,
wherein the pre-defined tearable portion is tearable from the layered dressing arrangement to reduce a surface area of the layered dressing arrangement.

2. The modular medical dressing of claim 1, wherein the pre-defined tearable portion comprises a plurality of first tear lines connected to define a first tearable portion.

3. The modular medical dressing of claim 2, wherein the pre-defined tearable portion comprises a plurality of second tear lines spaced apart from the plurality of first tear lines and connected to define a second tearable portion.

4. The modular medical dressing of claim 1, wherein the pre-defined tearable portion is a perforation extending through at least a portion of each of the dressing layer, the release layer, and the backing layer.

5. The modular medical dressing of claim 1, wherein the dressing layer is transparent.

6. The modular medical dressing of claim 1, wherein the dressing layer comprises a catheter access slot.

7. The modular medical dressing of claim 1, wherein the second surface of the dressing layer comprises an adhesive for adhering the dressing layer to an anatomical structure.

8. The modular medical dressing of claim 1, wherein the dressing layer is made from a polyurethane material.

9. The modular medical dressing of claim 1, wherein the backing layer is made from a paper material.

10. The modular medical dressing of claim 1, wherein the release layer is made from a paper material and comprises an adhesive on a surface facing the first surface of the dressing layer.

11. The modular medical dressing of claim 1, wherein at least a portion of the release layer is configured for use as securement tape for securing a catheter to an anatomical structure.

12. A modular medical dressing comprising:
a layered dressing arrangement comprising a dressing layer having a first surface and a second surface and a release layer releasably adhered to the first surface; and
a pre-defined tearable portion formed on the layered dressing arrangement,
wherein the pre-defined tearable portion is tearable from the layered dressing arrangement to reduce a size of the layered dressing arrangement.

13. The modular medical dressing of claim 12, further comprising a backing layer made of paper and releasably adhered to the second surface of the dressing layer.

14. The modular medical dressing of claim 12, wherein the pre-defined tearable portion comprises a plurality of first tear lines connected to define a first tearable portion.

15. The modular medical dressing of claim 14, wherein the pre-defined tearable portion comprises a plurality of second tear lines spaced apart from the plurality of first tear lines and connected to define a second tearable portion.

16. The modular medical dressing of claim 12, wherein the pre-defined tearable portion is a perforation extending through at least a portion of each of the dressing layer, the release layer, and the backing layer.

17. The modular medical dressing of claim 12, wherein the dressing layer is transparent.

18. The modular medical dressing of claim 12, wherein the second surface of the dressing layer comprises an adhesive for adhering the dressing layer to an anatomical structure.

19. The modular medical dressing of claim 12, wherein the release layer is made from a paper material and comprises an adhesive on a surface facing the first surface of the dressing layer.

20. The modular medical dressing of claim 12, wherein a portion of the release layer is configured for use as securement tape for securing a catheter to an anatomical structure.
